(19) United States
(12) Patent Application Publication
(10) Pub. No.: US 2025/0246265 A1
(43) Pub. Date: Jul. 31, 2025

(54) METHODS AND SYSTEMS FOR DETERMINING COPY NUMBER VARIANT GENOTYPES

(71) Applicant: Illumina, Inc., San Diego, CA (US)

(72) Inventors: Vitor Ferreira Onuchic, San Diego, CA (US); Xiao Chen, Richland, WA (US); Shunhua Han, San Diego, CA (US)

(21) Appl. No.: 18/866,918

(22) PCT Filed: Jul. 5, 2023
(86) PCT No.: PCT/US2023/026935

§ 371 Date: Nov. 18, 2024

Related U.S. Application Data
(60) Provisional application No. 63/367,888, filed on Jul. 7, 2022.

(51) Int. Cl. G16B 30/00; G16B 40/20

(52) U.S. Cl. CPC G16B 30/00; G16B 40/20

(57) ABSTRACT

Disclosed herein are systems, devices, and methods for identifying recombinant variants (such as deletion or duplication variants) of genes such as HBA1 gene and HBA2 gene, the copy numbers of HBA1 and/or HBA2, and a copy number variant genotype. Also disclosed herein are systems, devices, and methods for detecting one or more single-nucleotide variants or indels in a HBA1/2 region in a nucleic acid sample.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims priority to U.S. Provisional Application No. 63/367,888, filed Jul. 7, 2022, and entitled “METHODS AND SYSTEMS FOR IDENTIFYING GENOTYPES,” which is hereby incorporated by reference in its entirety.

### BACKGROUND

### Field

The disclosed technology relates to the field of nucleic acid sequencing. More particularly, the disclosed technology relates to determining a HBA1/2 copy number variant genotype in a nucleic acid sample.

### Description of the Related Art

Mutations or copy number variation in HBA1/2 can result in α-thalassemia, one of the world's most common human monogenetic diseases with a 5% carrier frequency worldwide. Approximately 95% of a-thalassemia cases result from gene deletion(s) rather than non-deletional mutations. HBA1 and HBA2 genes are at least 97% homologous. Common one- and two-copy HBA1/2 deletions in a-thalassemia include the α3.7 deletion, the α4.2 deletion, the Southeast Asian (SEA) deletion, and the Mediterranean (MED) deletion.

About 95% of alpha-thalassemia cases result from gene deletion(s) rather than non-deletional variants. For example, FIG. 1A illustrates potential gene deletion(s) and non-deletional variants and the resulting phenotype. Detecting alpha-thalassemia variants from standard whole genome sequencing (WGS) data can be a challenge, in part due to high homology between HBA1 and HBA2 gene regions that results in ambiguous read alignments. Determination of variants in HBA1/2 copy number variant genotypes can be complicated by the high sequence similarity observed between the two genes. For example, sequence reads of the HBA1 or HBA2 genes can, in some cases, be misaligned to the wrong gene or can be mapped with equal confidence to both genes, leading to low mapping quality. This may make sequence assembly through the HBA1 and HBA2 genes inaccurate and may lead to inaccurate determination of HBA1 and/or HBA2 copy number.

Additionally, conventional variant detection methods may struggle to accurately detect deletions such as α3.7 and α4.2 deletions because these deletions may have boundaries which fall in segmental duplication regions such as regions X, Y, and Z described herein. For example, a deletion may happen in a region of a segmental duplication. Conventional methods of variant detection may discard or not leverage sequence reads from segmental duplications, thereby causing a deletion such as an α3.7 deletion or an α4.2 deletion to go undetected.

### SUMMARY

In one aspect, disclosed herein are computer-implemented methods of determining a HBA1/2 copy number variant genotype in a nucleic acid sample. In some embodiments, the methods include: determining sequence reads from the nucleic acid sample; counting sequence reads which align to diploid regions in a human genome within the nucleic acid sample; counting sequence reads which align to a target region of one or more target regions adjacent to the locations of a HBA1 gene and a HBA2 gene in the human genome; and determining a HBA1/2 copy number variant genotype based on the count of the sequence reads which align to a target region of the one or more target regions as compared to the count of the sequence reads which align to the diploid regions in the human genome.

In some embodiments, determining a HBA1/2 copy number variant genotype includes estimating an integer copy number for each of the one or more target regions. In some embodiments, determining a HBA1/2 copy number variant genotype includes normalizing the count of the sequence reads which align to each target region by the count of the sequence reads which align to the diploid regions in the human genome to determine a float copy number for each of the one or more target regions.

In some embodiments, estimating an integer copy number for each of the one or more target regions further includes applying a Gaussian mixture model to the float copy number of the sequence reads which align to each target region. In some embodiments, the Gaussian mixture model comprises a pre-defined shift, prior, mean, or standard deviation as set forth in Table 3.

In some embodiments, the one or more target regions adjacent to the locations of the HBA1 and HBA2 genes in the human genome comprise a first upstream region upstream of the HBA2 gene and the HBA1 gene. In some embodiments, the one or more target regions adjacent to the locations of the HBA1 and HBA2 genes in the human genome further comprise a second upstream region upstream of the HBA2 gene and the HBA1 gene. In some embodiments, the one or more target regions adjacent to the locations of the HBA1 and HBA2 genes in the human genome comprise an intergenic region in between the HBA2 and HBA1 genes, or a downstream region downstream of the HBA2 and HBA1 genes. In some embodiments, the one or more target regions comprise a first and second upstream region upstream of the HBA2 gene and the HBA1 gene, an intergenic region in between the HBA2 and HBA1 genes, and a downstream region downstream of the HBA2 and HBA1 genes.

In some embodiments, sequence reads align to each of the one or more target regions with an alignment MAPQ score of at least 30. In some embodiments, 9 the first upstream region flanks a segmental duplication region X upstream of the HBA2 gene. In some embodiments, the second upstream region corresponds to a region within an α4.2 deletion event. In some embodiments, the second upstream region flanks a segmental duplication region Z upstream of the HBA2 gene. In some embodiments, the intergenic region corresponds to a region within an α3.7 deletion event. In some embodiments, the intergenic region flanks a segmental duplication region Z upstream of the HBA1 gene. In some embodiments, the first upstream region, the second upstream region, the intergenic region, and the downstream region correspond to regions within a deletion event in cis of both HBA1 and HBA2.

In some embodiments, the first upstream region has the coordinates chr16:167503-169503 in reference genome hg38, the second upstream region has the coordinates chr16:170263-171875 in reference genome hg38, the intergenic region has the coordinates chr16:174519-175845 in reference genome hg38, or the downstream region has the coordinates chr16:178002-180501 in reference genome hg38.

In some embodiments, determining a HBA1/2 copy number variant genotype comprises determining an aaa^{3.7}/aa genotype, an aaa^{4.2}/aa genotype, an aa/aa genotype, an -a^{3.7}/aa genotype, an -a^{4.2}/aa genotype, an --/aaa^{3.7} genotype, an --/aaa^{4.2} genotype, an -a^{3.7}/-a^{3.7} genotype, an -a^{4.2}/-a^{4.2} genotype, an -a^{3.7}/-a^{4.2} genotype, an --/aa genotype, an --/a^{3.7} genotype, an --/a^{4.2} genotype, or a --/-- genotype.

In another aspect, disclosed herein are computer-implemented methods of detecting one or more single-nucleotide variants or indels in a HBAP1/2 region in a nucleic acid sample. In some embodiments, the methods include: determining sequence reads from the nucleic acid sample; obtaining sequence reads which align to a site of a single-nucleotide variant or indel in a HBA1 gene or a HBA2 gene of a human genome in the nucleic acid sample; counting sequence reads which contain a base corresponding to an alternative allele at the site of the single-nucleotide variant or indel, wherein counting sequence reads comprises counting sequence reads which align to the HBA1 gene and sequence reads which align to the HBA2 gene; and creating a digital file including a variant call corresponding to the single-nucleotide variant or indel, wherein the variant call is not specific to the HBA1 gene or the HBA2 gene.

In some embodiments, the single-nucleotide variant or indel is HBA2_c.60del, HBA2_c.69C>T, HBA2_c.95+2_95+6delTGAGG, HBA2_c.95+1G>A, HBA1_c.179G>A, HBA2_c.377T>C, HBA2_c.427T>C, HBA2_c.427T>G, HBA2_c.429A>T, HBA2_c.*92A>G, HBA2_c.428A>C, HBA2_c.314G>A, HBA2_c.379G>A, HBA2_c.179G>A, HBA2_c.75T>G, HBA1_c.96-1G>A, HBA1_c.358C>T, or HBA2_c.*94A>G.

In another aspect, disclosed herein are electronic systems for determining a HBA1/2 copy number variant genotype in a nucleic acid sample. In some embodiments, the electronic systems include a processor configured to perform a method comprising: determining sequence reads from the nucleic acid sample; counting sequence reads which align to diploid regions in a human genome within the nucleic acid sample; counting sequence reads which align to a target region of one or more target regions adjacent to the locations of a HBA1 gene and a HBA2 gene in the human genome; and determining a HBA1/2 copy number variant genotype based on the count of the sequence reads which align to a target region of the one or more target regions as compared to the count of the sequence reads which align to the diploid regions in the human genome.

In some embodiments, determining a HBA1/2 copy number variant genotype comprises estimating an integer copy number for each of the one or more target regions. In some embodiments, determining a HBA1/2 copy number variant genotype comprises normalizing the count of the sequence reads which align to each target region by the count of the sequence reads which align to the diploid regions in the human genome to determine a float copy number for each of the one or more target regions. In some embodiments, estimating an integer copy number for each of the one or more target regions further comprises applying a Gaussian mixture model to the float copy number of the sequence reads which align to each target region.

In another aspect, disclosed herein are electronic systems for detecting one or more single-nucleotide variants or indels in a HBA1/2 region in a nucleic acid sample. In some embodiments, the electronic systems include a processor configured to perform a method comprising: determining sequence reads from the nucleic acid sample; obtaining sequence reads which align to a site of a single-nucleotide variant or indel in a HBA1 gene or a HBA2 gene of a human genome in the nucleic acid sample; counting sequence reads which contain a base corresponding to an alternative allele at the site of the single-nucleotide variant or indel, wherein counting sequence reads comprises counting sequence reads which align to the HBA1 gene and sequence reads which align to the HBA2 gene; and creating a digital file including a variant call corresponding to the single-nucleotide variant or indel, wherein the variant call is not specific to the HBA1 gene or the HBA2 gene.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A illustrates gene deletion(s) and non-deletional variants that can result in α-thalassemia.

FIG. 1B schematically illustrates a HBA1/2 region.

FIG. 1C schematically illustrates a HBA1/2 region.

FIG. 2A is a block diagram that schematically illustrates methods of determining a HBA1/2 copy number variant genotype in a nucleic acid sample.

FIG. 2B is a block diagram that further schematically illustrates a process of determining a HBA1/2 copy number variant genotype.

FIG. 3A is a block diagram of an exemplary sequencing system that may be used to perform the disclosed methods.

FIG. 3B is a block diagram of an exemplary computing device that may be used in connection with the exemplary sequencing system of FIG. 3A.

FIG. 4 schematically illustrates Mendelian inheritance of a HBA1/2 copy number variant genotype.

### DETAILED DESCRIPTION

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

One embodiment of the invention is a targeted gene calling approach for detecting deletional and/or non-deletional variants of HBA1/2 genes from sequence reads, such as standard whole genome sequencing data. In some embodiments, the use of one or more target regions as further described herein provides for the detection of clinically relevant HBA1/2 copy number variants, including one-copy deletions such as α3.7 and α4.2, and two-copy deletions in cis such as SEA. Embodiments of the present disclosure provide for the determination of multiple haplotypes of copy number genotypes in HBA1/2, such as -a^{3.7}/aa that represents a heterozygous α3.7 deletion.

### Overview

Described herein are methods and systems for detecting a HBA1/2 copy number variant genotype in a nucleic acid sample taken from a subject. The disclosed systems and methods for of determining a HBA1/2 copy number variant genotype in a nucleic acid sample have improved specificity and sensitivity of determining a HBA1/2 copy number variant genotypes and of variant calling in the HBA1 and/or HBA2 regions in the nucleic acid sample. In some embodiments, the disclosed systems and methods solve the technical problem of inaccurate HBA1 and HBA2 copy number determination due to ambiguous sequence read alignments to the HBA1 gene and the HBA2 gene due to high sequence similarity.

In some embodiments, the disclosed systems and methods include determining sequence reads from the nucleic acid sample. Once sequence reads are determined, the sequence reads may be aligned to a reference genome. The method may further include counting sequence reads which align to diploid regions in a human genome within the nucleic acid sample. For example, the diploid regions may be regions which are generally diploid in a nucleic acid sample from a human.

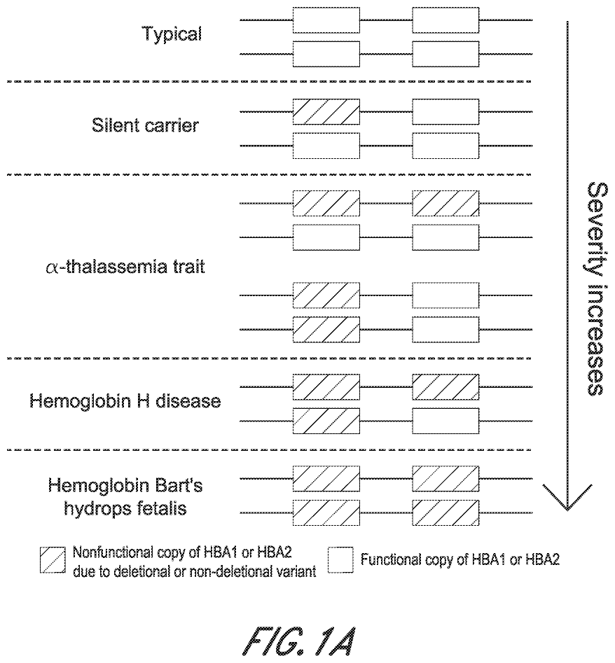

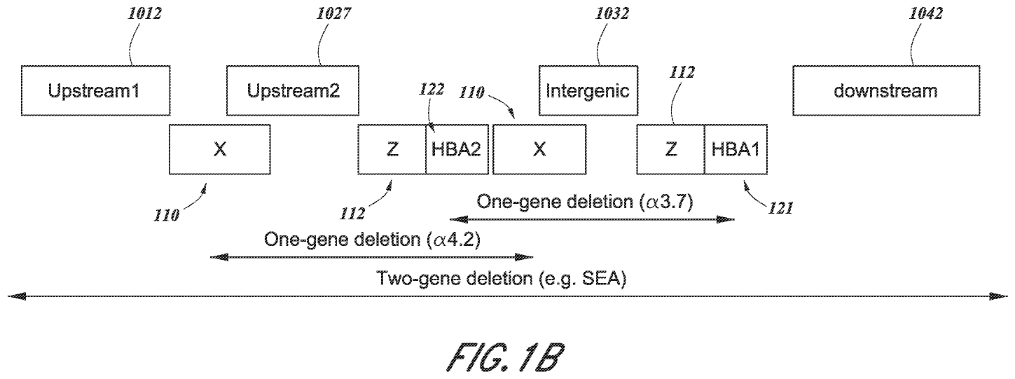

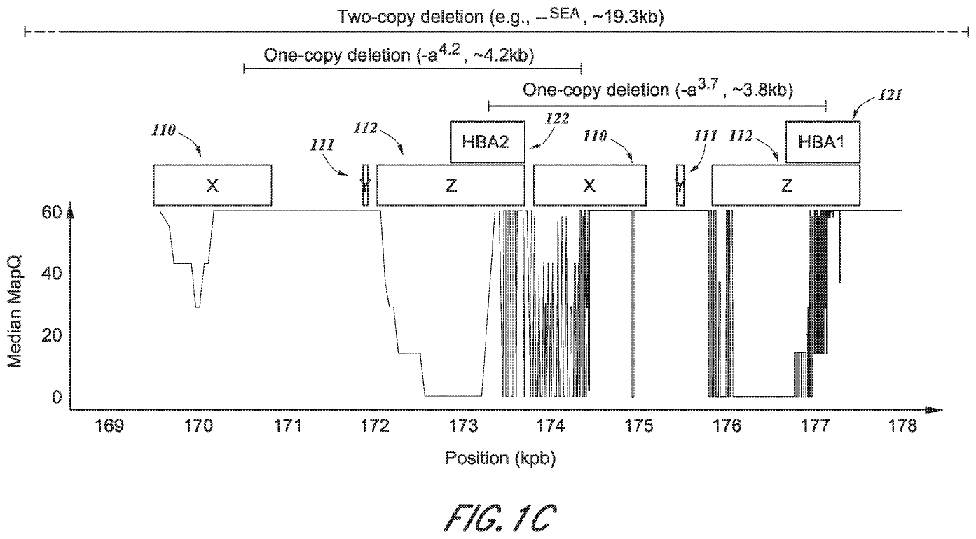

The disclosed methods and systems may then count the sequence reads which align to one or more target regions adjacent to the locations of the HBA1 and HBA2 genes in the human genome. The target regions may include a first and/or second upstream region upstream of the HBA2 gene and the HBA1 gene, an intergenic region in between the HBA2 and HBA1 genes, and/or a downstream region downstream of the HBA2 and HBA1 genes. In some embodiments, the first upstream region 1012, the second upstream region 1027, the intergenic region 1032, and the downstream region 1042 may have genetic locations substantially as shown in FIG. 1B. FIG. 1C depicts, among other things, segmental duplication region X 110, segmental duplication region Y 111, and segmental duplication region Z 112 near the HBA2 gene locus 122 and HBA1 gene locus 121. These regions X, Y, and Z are well known and studied to those of skill in the art and are described in, for example, Farashi and Harteveld, Molecular basis of α-thalassemia, Blood Cells, Molecules, and Diseases, 70:43-53 (2018).

The disclosed systems and methods may then determine a HBA1/2 copy number variant genotype based on a count of the sequence reads which align to each of the one or more target regions as compared to the count of the sequence reads which align to the diploid regions in the human genome. For example, the disclosed systems and methods may estimate an integer copy number for each of the one or more target regions. For example, the disclosed systems and methods may normalize the count of the sequence reads which align to each target region by the count of the sequence reads which align to the diploid regions in the human genome, such as non-repetitive regions with stable diploid copy number in a population, to determine a float copy number for each of the one or more target regions. The disclosed systems and methods may apply a Gaussian mixture model to the float copy number of the sequence reads which align to each target region to estimate an integer copy number for each of the one or more target regions.

The disclosed systems and methods can improve the specificity, the percentage of true variants that are correctly detected, of single nucleotide polymorphisms (SNPs) and/or insertion/deletions (indels) associated with a HBA1/2 copy number variant genotype by 20%, 50%, 80%, 100% or more, for example by increasing true positive detection of variants due to a HBA1/2 copy number variant genotype.

### Definitions

Unless defined otherwise, technical and scientific terms used herein have the same meaning as commonly understood by one of ordinary skill in the art to which the present disclosure belongs. See, for example, Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “base” or “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

A “genome” refers to the complete genetic information of an organism or virus, expressed in nucleic acid sequences.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. In various embodiments, the reference sequence is significantly larger than the reads that are aligned to it. For example, it may be at least about 100 times larger, or at least about 1000 times larger, or at least about 10,000 times larger, or at least about 105 times larger, or at least about 106 times larger, or at least about 107 times larger. In one example, the reference sequence is that of a full-length genome. Such sequences may be referred to as genomic reference sequences. For example, the reference sequence can be a reference human genome sequence, such as hg19 or hg38. In another example, the reference sequence is limited to a specific human chromosome such as chromosome 13. In some embodiments, a reference Y chromosome is the Y chromosome sequence from human genome version hg19. Such sequences may be referred to as chromosome reference sequences. Other examples of reference sequences include genomes of other species, as well as chromosomes, sub-chromosomal regions (such as strands), etc., of any species. In various embodiments, the reference sequence is a consensus sequence or other combination derived from multiple individuals. However, in certain applications, the reference sequence may be taken from a particular individual.

The term “nucleic acid sample” herein refers to a sample, typically derived from a biological fluid, cell, tissue, organ, or organism, comprising a nucleic acid or a mixture of nucleic acids comprising at least one nucleic acid sequence that is to be screened for copy number variation. In certain embodiments the nucleic acid sample comprises at least one nucleic acid sequence whose copy number is suspected of having undergone variation. Such samples may include, but are not limited to sputum/oral fluid, amniotic fluid, blood, a blood fraction, or fine needle biopsy samples (such as surgical biopsy, fine needle biopsy, etc.), urine, peritoneal fluid, pleural fluid, and the like. Although the sample is often taken from a human subject (such as a patient), the sample may be from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (such as namely, a sample that is not subjected to any such pretreatment method(s)). Such “treated” or “processed” samples are still considered to be biological “test” samples with respect to the methods described herein.

The term “read” or “sequence read” (or sequencing reads) refer to a sequence obtained from a portion of a nucleic acid sample. A read may be represented by a string of nucleotides sequenced from any part or all of a nucleic acid molecule. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (such as at least about 25 bp) that can be used to identify a larger sequence or region, for example, that can be aligned and specifically assigned to a chromosome or genomic region or gene. For example, a sequence read may be a short string of nucleotides (such as 20-150 bases) sequenced from a nucleic acid fragment, a short string of nucleotides at one or both ends of a nucleic acid fragment, or the sequencing of the entire nucleic acid fragment that exists in the biological sample. Sequence reads may be obtained by any method known in the art. For example, a sequence read may be obtained in a variety of ways, such as using sequencing techniques or using probes, such as in hybridization arrays or capture probes, or amplification techniques, such as the polymerase chain reaction (PCR) or linear amplification using a single primer or isothermal amplification. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

The term “sequencing depth,” as used herein, generally refers to the number of times a locus is covered by a sequence read aligned to the locus. The locus may be as small as a nucleotide, or as large as a chromosome arm, or as large as the entire genome. Sequencing depth can be expressed as 50×, 100×, etc., where “×” refers to the number of times a locus is covered with a sequence read. Sequencing depth can also be applied to multiple loci, or the whole genome, in which case x can refer to the mean number of times the loci or the haploid genome, or the whole genome, respectively, is sequenced. When a mean depth is quoted, the actual depth for different loci included in the dataset spans over a range of values. Ultra-deep sequencing can refer to at least 100× in sequencing depth.

As used herein, the terms “aligned,” “alignment,” or “aligning” refer to the process of comparing a read or tag to a reference sequence and thereby determining the likelihood of the reference sequence contains the read sequence. If the reference sequence contains the read, the read may be mapped to the reference sequence or, in certain embodiments, to a particular location in the reference sequence. For example, the alignment of a read to the reference sequence for human chromosome 13 will tell the likelihood of the read is present in the reference sequence for chromosome 13. In some cases, an alignment additionally indicates a location where the read or tag maps to in the reference sequence. For example, if the reference sequence is the whole human genome sequence, an alignment may indicate that a read is present on chromosome 13, and may further indicate that the read is on a particular strand and/or site of chromosome 13. A “site” may be a unique position on a polynucleotide sequence or a reference genome (i.e. chromosome ID, chromosome position and orientation). In some embodiments, a site may provide a position for a residue, a sequence tag, or a segment on a sequence.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. The matching of a sequence read in aligning can be a 100% sequence match or less than 100% (non-perfect match).

Alignment may be performed by modifications and/or combinations of methods such as Burrows-Wheeler Aligner (BWA), iSAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM

The term “mapping” used herein refers to specifically assigning a sequence read to a larger sequence, such as a reference genome, by alignment.

A “genetic variation” or “genetic alteration” refers to a particular genotype present in certain individuals, and often a genetic variation is present in a statistically significant sub-population of individuals. The presence or absence of a genetic variance can be determined using a method or apparatus described herein. In certain embodiments, the presence or absence of one or more genetic variations is determined according to an outcome provided by methods and apparatuses described herein. In some embodiments, a genetic variation is a chromosome abnormality (such as aneuploidy), partial chromosome abnormality or mosaicism, each of which is described in greater detail herein. Non-limiting examples of genetic variations include one or more deletions (such as micro-deletions), duplications (such as micro-duplications), insertions, mutations, polymorphisms (such as single-nucleotide polymorphisms), fusions, repeats (such as short tandem repeats), distinct methylation sites, distinct methylation patterns, the like and combinations thereof. An insertion, repeat, deletion, duplication, mutation or polymorphism can be of any length, and in some embodiments, is about 1 base or base pair (bp) to about 250 megabases (Mb) in length. In some embodiments, an insertion, repeat, deletion, duplication, mutation or polymorphism is about 1 base or base pair (bp) to about 1,000 kilobases (kb) in length (for example about 10 bp, 50 bp, 100 bp, 500 bp, 1 kb, 5 kb, 10 kb, 50 kb, 100 kb, 500 kb, or 1000 kb in length).

A genetic variation is sometimes a deletion. In certain embodiments a deletion is a mutation (such as a genetic aberration) in which a part of a chromosome or a sequence of DNA is missing. A deletion is often the loss of genetic material. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can comprise a microdeletion. A deletion can comprise the deletion of a single base.

A genetic variation is sometimes a genetic duplication. In certain embodiments a duplication is a mutation (such as a genetic aberration) in which a part of a chromosome or a sequence of DNA is copied and inserted back into the genome. In certain embodiments a genetic duplication (i.e. duplication) is any duplication of a region of DNA. In some embodiments a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can comprise a microduplication. A duplication sometimes comprises one or more copies of a duplicated nucleic acid. A duplication sometimes is characterized as a genetic region repeated one or more times (such as repeated 1, 2, 3, 4, 5, 6, 7, 8, 9 or 10 times). Duplications can range from small regions (thousands of base pairs) to whole chromosomes in some instances. Duplications frequently occur as the result of an error in homologous recombination or due to a retrotransposon event. Duplications have been associated with certain types of proliferative diseases. Duplications can be characterized using genomic microarrays or comparative genetic hybridization (CGH).

A genetic variation is sometimes an insertion. An insertion is sometimes the addition of one or more nucleotide base pairs into a nucleic acid sequence. An insertion is sometimes a microinsertion. In certain embodiments an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (i.e., insertion) of nucleic acid of unknown origin into a genome, chromosome, or segment thereof. In certain embodiments an insertion comprises the addition (i.e., insertion) of a single base.

A genetic variation sometimes includes copy number variations, i.e., variations in the number of copies of a nucleic acid sequence present in a test sample in comparison with the copy number of the nucleic acid sequence present in a reference sample. In certain embodiments, the nucleic acid sequence is 1 kb or larger. In some cases, the nucleic acid sequence is a whole chromosome or significant portion thereof. A copy number variant may refer to the sequence of nucleic acid in which copy-number differences are found by comparison of a nucleic acid sequence of interest in test sample with an expected level of the nucleic acid sequence of interest. For example, the level of the nucleic acid sequence of interest in the test sample is compared to that present in a qualified sample. Copy number variants/variations may include deletions, including microdeletions, insertions, including microinsertions, duplications, multiplications, and translocations. CNVs encompass chromosomal aneuploidies and partial aneuploidies.

### Embodiments of Methods and Systems of Determining a HBA1/2 Copy Number Variant Genotype

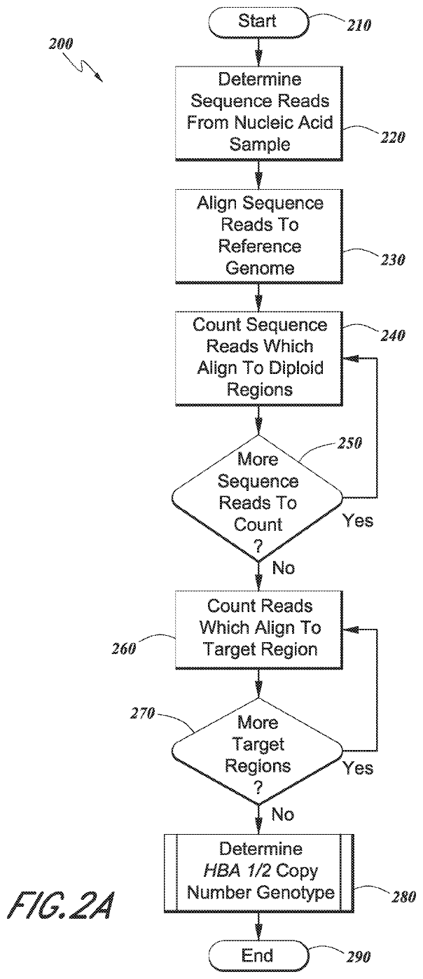

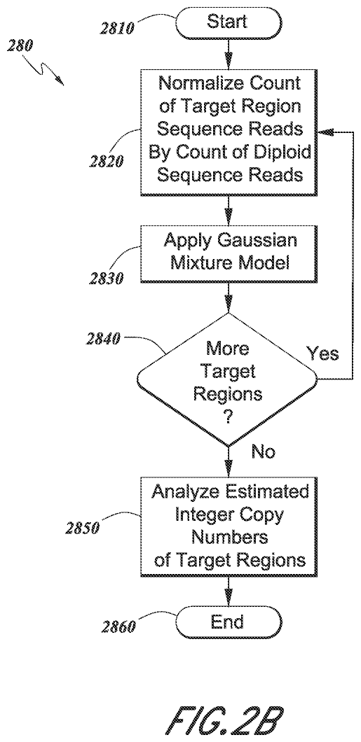

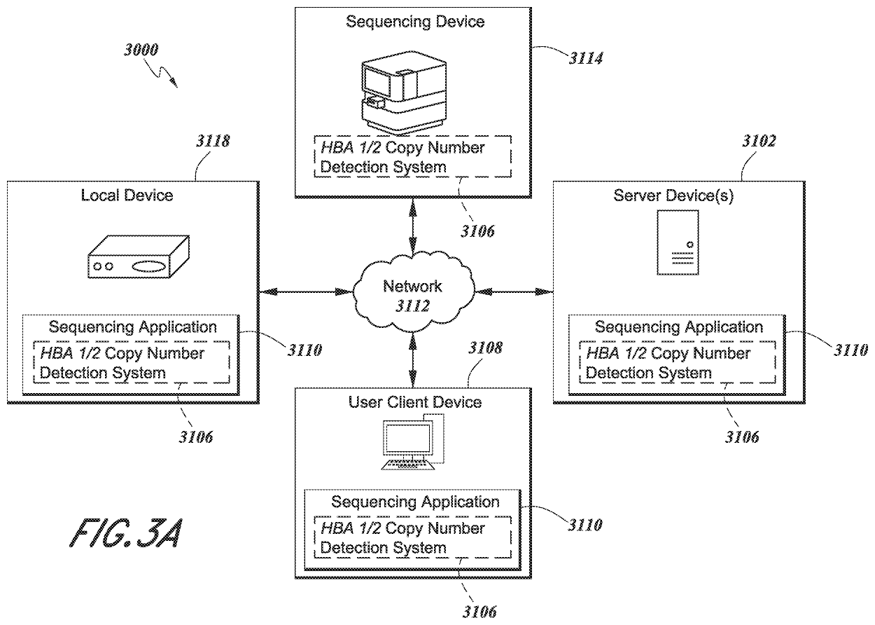

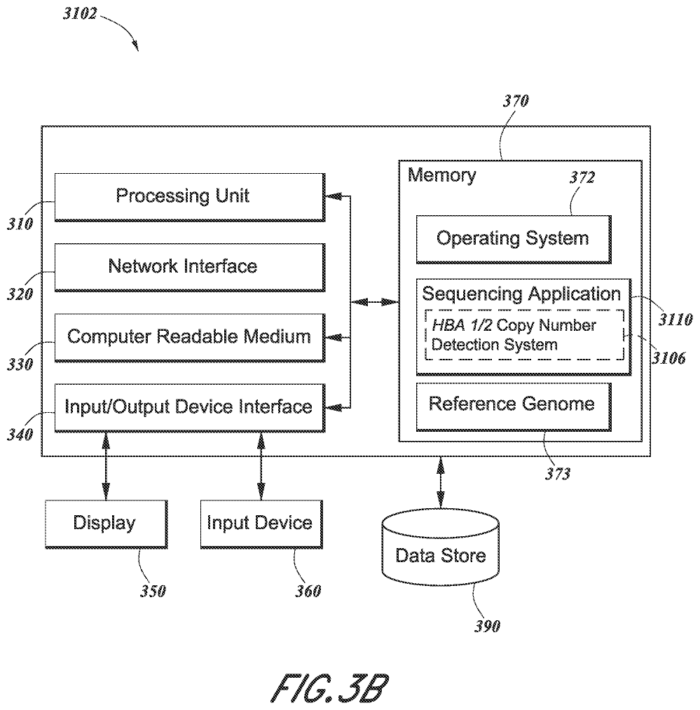

FIG. 2A is a block diagram that schematically illustrates an exemplary method 200 of determining a HBA1/2 copy number variant genotype in a nucleic acid sample. In some embodiments, the method 200 is implemented on a computer. The method 200 may be embodied in a set of executable program instructions stored on a computer-readable medium, such as one or more disk drives, of a computing system. For example, the server device 3102 shown in FIGS. 3A and 3B and described in greater detail below can execute a set of executable program instructions to implement the method 200. When the method 200 is initiated, the executable program instructions can be loaded into a memory, such as RAM, and executed by one or more processors of a server device 3102. Although the method 200 is described with respect to the server device 3102 shown in FIG. 3B, the description is illustrative only and is not intended to be limiting. In some embodiments, the method 200 or portions thereof may be performed serially or in parallel by multiple computing systems.

As shown in FIG. 2A, the method 200 for determining a HBA1/2 copy number variant genotype in a nucleic acid sample may start from start block 210. The method 200 may proceed to block 220, wherein sequence reads from a nucleic acid sample are determined. The method may next proceed to block 230, wherein sequence reads are aligned to a reference genome. Next, the method 200 may proceed to block 240, wherein sequence reads which align to diploid regions of a human genome within the nucleic acid sample are counted. The diploid regions may be non-repetitive regions with a stable diploid copy number in a population. Next the method 200 may proceed to decision state 250, wherein the system may decide whether there are more sequence reads which align to diploid regions to count. If there are additional sequence reads to count, the method 200 may return to block 240 and the method may proceed as previously described. If there are no additional sequence reads to count, the method 200 may proceed to block 260, wherein sequence reads which align to a target region adjacent to the locations of the HBA1 and HBA2 genes in the human genome are counted. The method may proceed to decision state 250, wherein the system may decide whether there are additional target regions for sequence read counting. If there are additional target regions, the method 200 may return to block 260 and the method may proceed as previously described. If there are no additional target regions, the method 200 may proceed to process block 280, wherein a HBA1/2 copy number genotype is determined. The process block 280 may be described in further detail with respect to FIG. 2B. The method 200 may end at end block 290.

FIG. 2B is a block diagram that further illustrates process block 280 described above, wherein a HBA1/2 copy number genotype is determined.

As shown in FIG. 2B, the method of process block 280, wherein a HBA1/2 copy number genotype is determined, may start from start block 2810. The method of process block 280 may proceed to block 2820, wherein the count of sequence reads aligned to a target region is normalized by the count of sequence reads aligned to diploid regions, thereby determining a float copy number for the target region. The method of process block 280 may proceed to block 2830, wherein a Gaussian mixture model is applied to the float copy number determined in block 2820, thereby determining an estimated copy number for the target region. The method of process block 280 may proceed to decision state 2840, wherein the system may decide if there are additional target regions for integer copy number estimation. If there are additional target regions, the method of process block 280 may return to block 2820, and the method may proceed as previously described. If there are no additional target regions, the method of process block 280 may proceed to block 2850, wherein the estimated integer copy numbers for the one or more target regions are analyzed. The method of process block 280 may end at end block 2860.

Determining Sequence Reads from the Nucleic Acid Sample

In some embodiments, the methods and systems disclosed herein include a step of determining sequence reads from a nucleic acid sample, for example block 220 of FIG. 2A. In some embodiments, the sequence reads are generated from a nucleic acid sample obtained from a subject.

Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA). Sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length each. For example, sequence reads are about 100 base pairs to about 1000 base pairs in length each. The sequence reads can comprise paired-end sequence reads. The sequence reads can comprise single-end sequence reads. The sequence reads can be generated by whole genome sequencing (WGS). The WGS can be clinical WGS (cWGS). The sample can comprise cells, cell-free DNA, cell-free fetal DNA, amniotic fluid, a blood sample, a biopsy sample, or a combination thereof.

In some embodiments, the sequence reads are aligned to a reference sequence, such as in block 230 of FIG. 2A. For example, sequence reads obtained from a sample may be aligned to one or more target regions adjacent to the locations of the HBA1 and HBA1/2 genes in the reference sequence. Sequence reads may also be aligned to diploid regions of a reference sequence as further described herein. In some embodiments, a computing system stores the first plurality of sequence reads in memory. The computing system may load the first plurality of sequence reads into memory.

In some embodiments, the sequence reads are obtained from a digital file containing sequencing information. In some embodiments, the digital file is on a computer storage medium (such as a computer hard drive, for example a spinning magnetic disk drive or a solid state drive). In some embodiments, the digital file is stored in the format of a BAM, SAM, CRAM, FASTQ, JSON, or VCF file.

### Counting Sequence Reads

In some embodiments, the disclosed systems and methods include a step of counting sequence reads which align to diploid regions in a human genome within the nucleic acid sample, for example block 240 of FIG. 2A. The diploid regions can include pre-selected regions across the genome of the subject which are measured to be consistently diploid across a population of nucleic acid samples. In some embodiments, the diploid regions are non-repetitive. For example, in some embodiments, alignment of sequence reads to the diploid regions is not ambiguous. For example, in some embodiments, sequence reads align to a diploid region with an alignment MAPQ score of at least 30, at least 40, at least 50, at least 60, at least 70, at least 80, or at least 90.

In some embodiments, the diploid regions can comprise about 100, about 500, about 1000, about 2000, about 3000, about 4000 or more pre-selected regions across the genome of the subject. In some embodiments, the length of a diploid region is about 100 bp, about 500 bp, about 1000 bp, about 2000 bp, about 3000 bp, about 5000 bp, or more, or a range constructed from any of the aforementioned values. In some embodiments, the length of a diploid region is about 2 kb. For example, the diploid regions may be randomly selected from the genome for stable coverage across population samples to infer the sequencing depth and capture GC bias. A system may determine if additional sequence reads which align to diploid regions remain to be counted, such as shown in decision state 250 of FIG. 2A.

In some embodiments, the disclosed systems and methods include a step of counting sequence reads which align to a target region of the one or more target regions adjacent to the locations of the HBA1 and HBA2 genes in the human genome, for example block 260 of FIG. 2A. In some embodiments, sequence reads are counted which align uniquely to a target region of the one or more target regions. In some embodiments, sequence reads align to a target region of the one or more target regions with an alignment MAPQ score of at least 30, at least 40, at least 50, at least 60, at least 70, at least 80, or at least 90. In some embodiments, the median MAPQ in each of the one or more target regions is about 60. In some embodiments, the system may count sequence reads which align to a first target region, and then determine whether additional target regions remain (such as a second, third, and/or fourth target region), such as is depicted in decision state 270 of FIG. 2A.

In some embodiments, the one or more target regions adjacent to the locations of the HBA1 and HBA2 genes in the human genome include a first upstream region upstream of the HBA2 gene and the HBA1 gene, a second upstream region upstream of the HBA2 gene and the HBA1 gene, an intergenic region in between the HBA2 and HBA1 genes, and/or a downstream region downstream of the HBA2 and HBA1 genes. In some embodiments, the first upstream region, the second upstream region, the intergenic region, and the downstream region have locations substantially as shown in FIG. 1B.

For example, in some embodiments, the one or more target regions adjacent to the locations of the HBA1 and HBA2 genes in the human genome include a first upstream region upstream of the HBA2 gene and the HBA1 gene. In some embodiments, the first upstream region flanks a segmental duplication region X upstream of the HBA2 gene. In some embodiments, the first upstream region has the coordinates of about chr16:167503-169503 in reference genome hg38 (for example, available at GenBank assembly accession GCA_000001405.15).

In some embodiments, the one or more target regions adjacent to the locations of the HBA1 and HBA2 genes in the human genome include a second upstream region upstream of the HBA2 gene and the HBA1 gene. In some embodiments, the second upstream region corresponds to a region within an α4.2 deletion event. In some embodiments, the second upstream region flanks a segmental duplication region Z upstream of the HBA2 gene. In some embodiments, the second upstream region has the coordinates of about chr16:170263-171875 in reference genome hg38.

In some embodiments, the one or more target regions adjacent to the locations of the HBA1 and HBA2 genes in the human genome include an intergenic region in between the HBA2 and HBA1 genes. In some embodiments, the intergenic region corresponds to a region within an α3.7 deletion event. In some embodiments, the intergenic region flanks a segmental duplication region Z upstream of the HBA1 gene. In some embodiments, the intergenic region has the coordinates of about chr16:174519-175845 in reference genome hg38.

In some embodiments, the one or more target regions adjacent to the locations of the HBA1 and HBA2 genes in the human genome include or a downstream region downstream of the HBA2 and HBA1 genes. In some embodiments, the downstream region flanks a downstream end of the HBA1 gene. In some embodiments, the downstream region has the coordinates of about chr16:178002-180501 in reference genome hg38.

In some embodiments, the first upstream region, the second upstream region, the intergenic region, and the downstream region correspond to regions within a deletion event in cis of both HBA1 and HBA2. For example, the deletion event in cis of both HBA1 and HBA2 may be a two-gene deletion such as a Southeast Asian (SEA) deletion or a Mediterranean (MED) deletion.

### Determining a Normalized and or GC-Corrected Copy Number

In some embodiments, the disclosed systems and methods include a step of determining a HBA1/2 copy number variant genotype based on the count of the sequence reads which align to a target region of the one or more target regions as compared to the count of the sequence reads which align to the diploid regions in the human genome, for example, process block 280 of FIG. 2A.

In some embodiments, determining a HBA1/2 copy number variant genotype includes determining a normalized count of sequence reads aligned to each of the one or more target regions. For example, in block 2820 of FIG. 2B, the count of sequence reads aligned to a target region is normalized by a count of sequence reads aligned to diploid regions. In some embodiments, determining a HBA1/2 copy number variant genotype includes a step of normalizing the sequence read count (such as of the target regions and/or diploid regions) by the length of the respective region. In some embodiments, determining the normalized count of the sequence reads aligned to the each of the one or more target regions comprises normalization using (1a) a depth of the sequence reads aligned to each of the one or more target regions, (1b) a length of each of the one or more target regions, (2a) a depth of sequence reads aligned to the diploid regions, and (2b) a length of each of the diploid regions.

In some embodiments, determining a HBA1/2 copy number variant genotype includes a step of normalizing the count of the sequence reads which align to each target region by the count of the sequence reads which align to the diploid regions in the human genome to determine a float copy number for each of the one or more target regions. For example, in some embodiments, the sequence read count (for example, a sequence read count normalized by length of the region) for each target region is pooled together with sequence read counts (for example, a sequence read count normalized by length of the region) for diploid regions including about 3,000 distinct 2 kb regions. Normalizing the count of sequence reads which align to a target region by the count of sequence reads which align to diploid regions may, in some embodiments, correct for bias in sequencing coverage due to variable GC content among different regions. For example, the count of sequence reads aligned to each of the one or more target regions may be corrected for GC content using sequence using (1) a GC content of each of the one or more target regions and (2) a GC content of each of diploid regions. In some embodiments, a normalized and/or GC-corrected copy number is determined for each of the one or more target regions. In some embodiments, the normalized and/or GC-corrected copy number is a float copy number, including a non-integer number such as 1.2, 2.4, etc.

### Determining an Estimated Integer Copy Number

In some embodiments, determining a HBA1/2 copy number variant genotype includes a step of estimating an integer copy number for each of the one or more target regions. In some embodiments, estimating an integer copy number for each of the one or more target regions further comprises applying a Gaussian mixture model to the float copy number of the sequence reads which align to each target region. For example, in block 2830 of FIG. 2B, a Gaussian mixture model is applied to a normalized count of sequence reads aligned to a target region.

In some embodiments, after determining a normalized and/or GC-corrected depth, an estimated integer copy number (CN) for the each of the one or more target regions is determined using a Gaussian mixture model (GMM). In some embodiments, the GMM includes pre-defined parameters such as shift, prior, mean, and standard deviation (sd). In some embodiments, a normalized and GC-corrected depth is first scaled by a shift value that corrects for alignment bias between target region and diploid regions. In some embodiments, the posterior probability of CN=i given scaled depth is then computed for i=0-6 based on the pre-trained mean, sd, and prior values from the Gaussian mixture model. In some embodiments, the integer copy number with highest posterior probability is then selected as candidate for the final integer copy number estimate.

In some embodiments, estimating the integer copy number comprises binning the normalized count of the sequence reads using a Gaussian mixture model. For example, a Gaussian mixture model may be used to infer the most likely copy number of a target region based on the observed normalized depth signal.

The estimated integer copy number can be, for example, 1, 2, 3, 4, 5, 6, 7, 8, 9, 10, or more copies. The Gaussian mixture model can comprise a one-dimensional Gaussian mixture model. The plurality of Gaussians of the Gaussian mixture model can represent integer copy numbers, for example, 0 to 5, 0 to 6, 0 to 7, 0 to 8, 0 to 9, 0 to 10, 0 to 11, 0 to 12, 0 to 13, 0 to 14, or 0 to 15. For example, the plurality of Gaussians of the Gaussian mixture model can represent integer copy numbers from 0 to 10. A mean of each of the plurality of Gaussians can be the integer copy number represented by the Gaussian. A mean of each of the plurality of Gaussians can be the integer copy number represented by the Gaussian (such as copy numbers of 1, 2, 3, 4, 5, 6, 7, 8, 9, 10, 11, 12, 13, 14, 15, or more). The standard deviation of a Gaussian can be or be about, for example, 0.1, 0.2, 0.3, 0.4, 0.5, 0.6, 0.7, 0.8, 0.9, 1, or more. The plurality of Gaussians of the Gaussian mixture model can comprise, for example, 5, 6, 7, 8, 9, 10, 11, 12, 13, 14, 15, 16, or more, Gaussians. For example, the plurality of Gaussians of the Gaussian mixture model can comprise 5 Gaussians.

To estimate an integer copy number, the computing system can determine the copy number using a Gaussian mixture model and a predetermined posterior probability threshold, given the normalized number of the sequence reads aligned to the target region. The predetermined posterior probability threshold can be, for example, 0.7, 0.75, 0.8, 0.85, 0.95, or more. In some embodiments, the predetermined posterior probability threshold is 0.95.

In some embodiments, the Gaussian mixture model (GMM) includes an optimized Gaussian mixture model. In some embodiments, the GMM parameters are trained based on an expectation maximization algorithm. For example, optimized parameters may be trained by starting with three randomly placed Gaussians (with parameters randomly initialized). Float copy numbers obtained as described herein from many nucleic acid samples may be used as training data for the Gaussian Mixture Model. For example, for each float copy number x for a given sample, P(x|CN=1), P(x|CN=2), and P(x|CN=3) may be calculated. The sample integer copy number may then be reassigned to CN=k, which has highest posterior P(CN=k|x). The parameters of the GMM may then be adjusted to fit points assigned to them. The process may be iterated until the parameters reach convergence. In some embodiments, the converged parameters may be used in a Gaussian mixture model as described herein.

In some embodiments, the Gaussian mixture model includes optimized parameters for each of the one or more target regions. In some embodiments, for a first upstream target region, the Gaussian mixture model has a shift of about 1.029, a mean (2,3) of about 2:1.0 and about 3:1.5, a prior (0-4) of about 0:0.001, about 1:0.01, about 2:0.987, about 3:0.0005, and about 4:0.0005, and/or a standard deviation (2) of about 0.062. In some embodiments, for a second upstream target region, the Gaussian mixture model has a shift of about 1.02, a mean (2,3) of about 2:1.0 and about 3:1.5, a prior (0-4) of about 0:0.001, about 1:0.015, about 2:0.987, about 3:0.005, and about 4:0.0005, and/or a standard deviation (2) of about 0.0073. In some embodiments, for an intergenic target region, the Gaussian mixture model has a shift of about 0.966, a mean (2,3) of about 2:1.0 and about 3:1.476, a prior (0-4) of about 0:0.012, about 1:0.13, about 2:0.834, about 3:0.023, and about 4:0.0005, and/or a standard deviation (2) of about 0.0077. In some embodiments, for a downstream target region, the Gaussian mixture model has a shift of about 1.071, a mean (2,3) of about 2:1.0 and about 3:1.5, a prior (0-4) of about 0:0.001, about 1:0.01, about 2:0.987, about 3:0.001, and about 4:0.0005, and/or a standard deviation (2) of about 0.06.

In some embodiments, the probability of the estimated integer copy number is calculated as, for example, a quality check of the estimated integer copy number. In some embodiments, an estimated integer copy number is only determined if the posterior probability is greater than 0.95 and the p-value of scaled depth in the Gaussian distribution of candidate copy number is greater than 0.001. In some embodiments, a HBA1/2 copy number genotype is not determined if any of the one or more target regions does not have an estimated integer copy number that passes quality check.

In some embodiments, estimation of an integer copy number is iterated for each of the one or more target regions. For example, in decision state 2840, a system may determine if more target regions remain to be analyzed as previously described. For example, an estimated integer copy number may be determined for each of the one or more target regions based on determining a normalized, GC-corrected float copy number as described herein, and based on application of a Gaussian mixture model as described herein.

### Determining a HBA1/2 Copy Number Variant Genotype

In some embodiments, estimated integer copy numbers for each of the one or more target regions are accumulated and compared to determine a HBA1/2 copy number variant genotype. For example, the systems and methods may analyze estimated integer copy numbers of target regions, as depicted in block 2850 of FIG. 2B. For example, in some embodiments, a copy number genotype of HBA1/2 is deterministically produced based on the estimated integer copy number estimates for each one of four target regions.

In some embodiments, determining a HBA1/2 copy number variant genotype comprises determining an aaa^{3.7}/aa genotype, an aaa^{4.2}/aa genotype, an aa/aa genotype, an -a^{3.7}/aa genotype, an -a^{4.2}/aa genotype, an --/aaa^{3.7} genotype, an --/aaa^{4.2} genotype, an -a^{3.7}/-a^{3.7} genotype, an -a^{4.2}/-a^{4.2} genotype, an -a^{3.7}/-a^{4.2} genotype, an --/aa genotype, an --/a^{3.7} genotype, an --/a^{4.2} genotype, or a --/-- genotype.

For example, the following table represents the copy number genotype of HBA1/2 that may be determined based on estimated integer copy numbers for each of four target regions (a first and second upstream region, an intergenic region, and a downstream region). In the table below, interpretation is research use only (RUO).

TABLE 1

| Upstream 1 | Upstream 2 | Intergenic | Downstream | Copy number | RUO |
|---|---|---|---|---|---|
| CN | CN | CN | CN | genotype | Interpretation |
| 2 | 2 | 3 | 2 | aaa^{3.7}/aa | Alpha-globin |
| | | | | | triplication |
| 2 | 3 | 2 | 2 | aaa^{4.2}/aa | Alpha-globin |
| | | | | | triplication |
| 2 | 2 | 2 | 2 | aa/aa | Normal |
| 2 | 2 | 1 | 2 | -a^{3.7}/aa | Silent Carrier |
| 2 | 1 | 2 | 2 | -a^{4.2}/aa | Silent Carrier |
| 1 | 1 | 2 | 1 | --/aaa^{3.7} | Carrier* |
| 1 | 2 | 1 | 1 | --/aaa^{4.2} | Carrier* |
| 2 | 2 | 0 | 2 | -a^{3.7}/-a^{3.7} | Carrier |
| 2 | 0 | 2 | 2 | -a^{4.2}/-a^{4.2} | Carrier |
| 2 | 1 | 1 | 2 | -a^{3.7}/-a^{4.2} | Carrier |
| 1 | 1 | 1 | 1 | --/aa | Carrier |
| 1 | 1 | 0 | 1 | --/-a^{3.7} | HbH |
| 1 | 0 | 1 | 1 | --/-a^{4.2} | HbH |
| 0 | 0 | 0 | 0 | --/-- | Hb Bart's |

In some embodiments, the methods and systems disclosed herein further includes a step of making a variant call for a HBA1/2 copy number variant. In some embodiments, the variant call includes a copy number genotype, including two or more copy number alleles.

In some embodiments, the methods and systems disclosed herein further include a step of creating a digital file including a variant call. In some embodiments, the file includes an estimated integer copy number for each of the one or more target regions, a float copy number for each of the one or more target regions, and a copy number genotype. In some embodiments, the digital file is on a computer storage medium (such as a computer hard drive, for example a spinning magnetic disk drive or a solid state drive). In some embodiments, the digital file is stored in the format of a BAM, SAM, CRAM, FASTQ, JSON, or VCF file. In some embodiments, the digital file is a VCF file or a JSON file.

### Method of Detecting Variants in a HBA1/2 Region

In another aspect, disclosed herein are methods and systems of detecting one or more single-nucleotide variants or indels in a HBA1/2 region in a nucleic acid sample. In some embodiments, the methods and systems determine sequence reads from the nucleic acid sample. For example, sequence reads may be determined as previously described herein with reference to methods and systems of determining a HBA1/2 copy number variant genotype.

In some embodiments, the methods and systems obtain sequence reads which align to a site of a single-nucleotide variant or indel in a HBA1 gene or a HBA2 gene of a human genome in the nucleic acid sample. For example, sequence reads may be aligned to a reference genome as previously described herein with reference to methods and systems of determining a HBA1/2 copy number variant genotype. In some embodiments, the sequence reads are derived from short-read sequencing. In some embodiments, the sequence reads are about 75 bp to about 500 bp in length. In other embodiments, the sequence reads are 200 bp to about 400 bp in length.

In some embodiments, the methods and systems count sequence reads which contain a base corresponding to an alternative allele at the site of the single-nucleotide variant or indel. In some embodiments, counting sequence reads comprises counting both sequence reads which align to the HBA1 gene (and which include the site of the single-nucleotide variant or indel) and sequence reads which align to the HBA2 gene (and which include the site of the single-nucleotide variant or indel). In some embodiments, the sequence read count may be normalized and GC-corrected as previously described herein with reference to methods and systems of determining a HBA1-2 copy number variant genotype.

In some embodiments, the methods and systems create a digital file including a variant call corresponding to the single-nucleotide variant or indel (collectively, “small variant”). In some embodiments, the small variant will be reported if a significant portion of sequence reads support the alternative allele. For example, the small variant may be reported if about 10% or more, about 20% or more, about 30% or more, about 40% or more, about 50% or more, about 60% or more, about 70% or more, or about 80% or more, or about 90% or more sequence reads which cover the small variant contain a basecall corresponding to an alternative allele at the site of the small variant, as compared to a reference allele at the site. In some embodiments, the small variant may be reported if one or more, two or more, three or more, four or more, five or more, six or more, seven or more, eight or more, nine or more, or ten or more sequence reads contain an alternative allele at the site of the variant.

In some embodiments, sequence reads which include an alternative allele, and sequence reads which contain a reference allele, are counted. In some embodiments, an integer copy number is estimated for an alternative or variant allele based on a) a combined count of sequence reads covering corresponding positions of the small variant in HBA1 and HBA2, b) a count of reads supporting reference alleles, and c) a count of reads supporting alternative alleles.

In some embodiments, the variant call is not specific to the HBA1 gene or the HBA2 gene. For example, in some embodiments, the variant call is not assigned to HBA1 or HBA2 or phased into one of the candidate haplotypes described further herein. In some embodiments, a small variant may be farther than one sequence read length (such as farther than 100 bp, 150 bp, 200 bp, 250 bp, 300 bp, 350 bp, or more) away from the one or more target regions described herein. In some embodiments, making a variant call ambiguous to HBA1 or HBA2 advantageously allows a user to detect one or more single-nucleotide variants or indels in a HBA1/2 region in a nucleic acid sample while more efficiently using computing power and memory, as a detected small variant does not need to be phased into a candidate haplotype, and the methods and systems do not require that sequence reads are further analyzed to determine whether a small variant is assigned to HBA1 or HBA2. In some embodiments, detecting a small variant in region-ambiguous manner improves computational resource efficiency and enables high precision and recall on discovering the variant allele, as compared to de-novo small variant calling or calling a small variant and phasing the small variant into a region or a haplotype, which require a much more complex process, are much less computationally efficient, and potentially provide less precision or recall for the variant of interest.

In some embodiments, variant call ambiguous to HBA1 or HBA2 advantageously allows a user to detect a small variant using short-read sequencing. Without being bound by theory, in some embodiments, short-read sequencing reads (such as sequence reads that include about 75-500 bp) over the HBA1 or HBA2 genes do not contain enough information to uniquely place the small variant and the user does not necessarily need to know the unique placement of the variant. In some embodiments, an advantage of making a region-ambiguous call is that the user avoids the need to perform more extensive sequencing assays such as long-read sequencing assays. The information required can be obtained from the same whole genome sequencing (WGS) assay used to variant call the rest of the genome.

In some embodiments, once a variant call ambiguous to HBA1 or HBA2 has been made, the placement of the single-nucleotide variant or indel in the HBA1 gene or the HBA2 gene can be confirmed with orthogonal (long-read) sequencing methods known to those of skill in the art. For example, after a single-nucleotide variant or indel is detected in a manner not specific to the HBA1 gene or the HBA2 gene, additional sequencing such as orthogonal techniques are used to confirm the variant call and/or phase the variant into regions.

In some embodiments, the single-nucleotide variant or indel includes a variant listed in the table below.

TABLE 2

| ClinVar Variation ID | Variant |
|---|---|
| 280127 | HBA2_c.60del |
| 375746 | HBA2_c.95 + 2_95 + 6delTGAGG |
| 15624 | HBA2_c.427T > C |
| 15627 | HBA2_c.428A > C |
| 15647 | HBA2_c.*92A > G |
| 15652 | HBA2_c.429A > T |
| 15656 | HBA2_c.314G > A |
| 15662 | HBA2_c.379G > A |
| 15687 | HBA2_c.69C > T |
| 15690 | HBA2_c.377T > C |
| 15849 | HBA1_c.179G > A |
| 375749 | HBA2_c.*94A > G |
| 439126 | HBA2_c.95 + 1G > A |
| 439112 | HBA2_c.179G > A |
| 618674 | HBA2_c.75T > G |
| 801169 | HBA1_c.96 − 1G > A |
| 811900 | HBA1_c.358C > T |
| 15653 | HBA2_c.427T > G |

In some embodiments, the methods and systems disclosed herein further include a step of creating a digital file including a variant call. In some embodiments, the file includes, for each single-nucleotide variant or indel, a reference for the small variant, a count of sequence reads supporting an alternative allele, and a count of sequence reads supporting a reference allele. In some embodiments, the digital file is on a computer storage medium (such as a computer hard drive, for example a spinning magnetic disk drive or a solid state drive). In some embodiments, the digital file is stored in the format of a BAM, SAM, CRAM, FASTQ, JSON, or VCF file. In some embodiments, the digital file is a VCF file or a JSON file.

### Embodiments of Sequencing Systems

FIG. 3A illustrates a diagram of an environment in which a HBA1/2 copy number detection system can operate in accordance with one or more implementations. The following paragraphs describe the HBA1/2 copy number detection system with respect to illustrative figures that portray example implementations and embodiments. For example, FIG. 3A illustrates a schematic diagram of a computing system 3000 in which a HBA1/2 copy number detection system 3106 operates in accordance with one or more implementations. As illustrated, the computing system 3000 includes one or more server device(s) 3102 connected to a user client device 3108, a local device 3118, and a sequencing device 3114 via a network 3112. The network 3112 can comprise any suitable network over which computing devices can communicate.

As shown in FIG. 3A, the computing system 3000 includes the server device(s) 3102. In various implementations, the server device(s) 3102 may generate, receive, analyze, store, and transmit digital data, such as data for nucleobase calls or sequenced nucleic-acid polymers. In some implementations, the server device(s) 3102 receive various data from the sequencing device 3114, such as data from a sample genome and/or sequence reads. The server device(s) 3102 may also communicate with the user client device 3108. In particular, the server device(s) 3102 can send data for sequence reads, direct nucleobase calls, nucleobase calls, and/or sequencing metrics to the user client device 3108.

As shown, the server device(s) 3102 includes a sequencing application 3110. In general, the sequencing application 3110 analyzes the data (such as call data) received from the sequencing device 3114 or elsewhere to determine nucleobase sequences for nucleic-acid polymers. For example, the sequencing application 3110 can receive raw data from the sequencing device 3114 and determine a nucleobase sequence for a sample genome or a nucleic-acid segment. In some implementations, the sequencing application 3110 determines the sequences of nucleobases in DNA and/or RNA segments or oligonucleotides.

As also shown, the sequencing application 3110 includes the HBA1/2 copy number detection system 3106. As described below, the HBA1/2 copy number detection system 3106 can determine a HBA1/2 copy number variant genotype in a nucleic acid sample. For example, in some embodiments, the HBA1/2 copy number detection system 3106 receives sequence reads obtained from a nucleic acid sample. The HBA1/2 copy number detection system 3106 further counts sequence reads which align to diploid regions in a human genome within the nucleic acid sample. The HBA1/2 copy number detection system 3106 further counts sequence reads which align to a target region of one or more target regions adjacent to the locations of a HBA1 gene and a HBA2 gene in the human genome. The HBA1/2 copy number detection system 3106 can determine a HBA1/2 copy number variant genotype based on the count of the sequence reads which align to a target region of the one or more target regions as compared to the count of the sequence reads which align to the diploid regions in the human genome.

Moreover, while the HBA1/2 copy number detection system 3106 is described being implemented on the server device(s) 3102, as part of the sequencing application 3110, in some implementations, the HBA1/2 copy number detection system 3106 is implemented by (such as located entirely or in part) on the user client device 3108, the sequencing device 3114, and/or the local device 3118. As mentioned, in some implementations, the HBA1/2 copy number detection system 3106 is implemented by one or more other components of the computing system 3000, such as the sequencing device 3114. In particular, the HBA1/2 copy number detection system 3106 can be implemented in a variety of different ways across the server device(s) 3102, the network 3112, the user client device 3108, the local device 3118, and the sequencing device 3114.

As further shown in FIG. 3A, the computing system 3000 includes the user client device 3108. In various implementations, the user client device 3108 can generate, store, receive, and send digital data. In particular, the user client device 3108 can receive the data from the sequencing device 3114. As further illustrated, the user client device 3108 includes a sequencing application 3110. The sequencing application 3110 may be a web application or a native application stored and executed on the user client device 3108 (e.g., a mobile application, desktop application, or web application). The sequencing application 3110 can receive data from the sequencing application 3110 and/or HBA1/2 copy number detection system 3106. For example, the user client device 3108 can receive variant call files and/or alignment files from the sequencing application 3110.

The sequencing application 3110 can also include instructions that (when executed) cause the user client device 3108 to receive data from the HBA1/2 copy number detection system 3106 and present data from the sequencing device 3114 and/or the server device(s) 3102. Furthermore, the sequencing application 3110 can instruct the user client device 3108 to display data for variant calls, such as nucleobase calls or an indication of a HBA1/2 copy number variant. Indeed, the user client device 3108 can display nucleobase call results for a genome sample and/or an indication of a predicted HBA1/2 copy number variant.

As further shown in FIG. 3A, the computing system 3000 includes the sequencing device 3114. In various implementations, the sequencing device 3114 can sequence a genomic sample or other nucleic-acid polymer. For example, the sequencing device 3114 analyzes nucleic-acid segments or oligonucleotides extracted from genomic samples to generate data either directly or indirectly on the sequencing device 3114. More particularly, the sequencing device 3114 receives and analyzes, within nucleotide-sample slides (such as flow cells), nucleic-acid sequences extracted from genomic samples. In one or more implementations, the sequencing device 3114 utilizes SBS to sequence a genomic sample or other nucleic-acid polymers. In addition to, or in the alternative to communicating across the network 3112, in some implementations, the sequencing device 3114 bypasses the network 3112 and communicates directly with the user client device 3108.

As further depicted in FIG. 3A, in some implementations, the server device(s) 3102 includes a distributed collection of servers, where the server device(s) 3102 include several server devices distributed across the network 3112 and located in the same or different physical locations. For instance, the server device(s) 3102 can be implemented, in whole or in part, on the local device 3118. To illustrate, the local device 3118 may implement the sequencing application 3110 and/or the HBA1/2 copy number detection system 3106. Further, the server device(s) 3102 and/or the local device 3118 can include a content server, an application server, a communication server, a web-hosting server, or another type of server.

The user client device 3108 illustrated in FIG. 3A can include various types of client devices. For example, in some implementations, the user client device 3108 includes non-mobile devices, such as desktop computers or servers, or other types of client devices. In various implementations, the user client device 3108 includes mobile devices, such as laptops, tablets, mobile telephones, or smartphones.

Though FIG. 3A illustrates the components of the computing system 3000 communicating via the network 3112, in certain implementations, the components of computing system 3000 can also communicate directly with each other, bypassing the network 3112. For instance, in some implementations, the user client device 3108 communicates directly with the sequencing device 3114. Additionally, in some implementations, the user client device 3108 communicates directly with the HBA1/2 copy number detection system 3106 and/or the server device(s) 3102. In some implementations, the user client device 3108 communicates directly with the local device 3118. Moreover, the HBA1/2 copy number detection system 3106 can access one or more databases housed on or accessed by the server device(s) 3102 or elsewhere in the computing system 3000.

FIG. 3B is a block diagram of an exemplary server device 3102 that may be used in connection with the illustrative sequencing system 3000 of FIG. 3A. The server device 3102 may be configured to determine a HBA1/2 copy number variant genotype in a nucleic acid sample. The general architecture of the server device 3102 depicted in FIG. 3B includes an arrangement of computer hardware and software components. The server device 3102 may include many more (or fewer) elements than those shown in FIG. 3B. It is not necessary, however, that all of these generally conventional elements be shown in order to provide an enabling disclosure. As illustrated, the server device 3102 includes a processing unit 310, a network interface 320, a computer readable medium drive 330, an input/output device interface 340, a display 350, and an input device 360, all of which may communicate with one another by way of a communication bus. The network interface 320 may provide connectivity to one or more networks or computing systems. The processing unit 310 may thus receive information and instructions from other computing systems or services via a network. The processing unit 310 may also communicate to and from memory 370 and further provide output information for an optional display 350 via the input/output device interface 340. The input/output device interface 340 may also accept input from the optional input device 360, such as a keyboard, mouse, digital pen, microphone, touch screen, gesture recognition system, voice recognition system, gamepad, accelerometer, gyroscope, or other input device.

The memory 370 may contain computer program instructions (grouped as modules or components in some embodiments) that the processing unit 310 executes in order to implement one or more embodiments. The memory 370 generally includes RAM, ROM and/or other persistent, auxiliary or non-transitory computer readable media. The memory 370 may store an operating system 372 that provides computer program instructions for use by the processing unit 310 in the general administration and operation of the server device 3102. The memory 370 may store a reference genome 373, such as for use by the sequencing application 3110. The memory 370 may further include computer program instructions and other information for implementing aspects of the present disclosure.

For example, in one embodiment, the memory 370 includes a sequencing application 3110, which may include a HBA1/2 copy number detection system 3106. The HBA1/2 copy number detection system 3106 can perform the methods disclosed herein. In addition, memory 370 may include or communicate with the data store 390 and/or one or more other data stores that store one or more inputs, one or more outputs, and/or one or more results (including intermediate results) of determining a HBA1/2 copy number variant genotype in a nucleic acid sample of the present disclosure, such the sequencing reads, the estimated copy number(s), and the variant call (for example, the detection of a HBA1/2 copy number variant) determined.

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD-ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (such as iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1

In the following example, GC-corrected and normalized depth for four target regions (Upstream 1, Upstream 2, Intergenic, and Downstream) from 2,407 unrelated samples from the 1000 Genomes Project was taken as input.

An expectation maximation process was used to determine optimized Gaussian mixture model (GMM) parameters. Three Gaussians (with parameters randomly initialized) were randomly placed. For each float copy number x for a given sample (and for each sample), P(x|CN=1), P(x|CN=2), and P(x|CN=3) was calculated to obtain a sample integer copy number. The sample integer copy number was then reassigned to CN=k, which has highest posterior P(CN=k|x). The parameters were then adjusted to fit points assigned to them. The process was iterated until the parameters reached convergence. The obtained parameters are described below in the following table.

TABLE 3

| Region | Parameter | Value | | | | |
|---|---|---|---|---|---|---|
| Upstream 1 | shift | 1.029 | | | | |
| Upstream 1 | mean (2, 3) | 2:1.0 | 3:1.5 | | | |
| Upstream 1 | prior (0-4) | 0:0.001 | 1:0.01 | 2:0.987 | 3:0.0005 | 4:0.0005 |
| Upstream 1 | sd (2) | 0.062 | | | | |
| Upstream 2 | shift | 1.02 | | | | |
| Upstream 2 | mean (2, 3) | 2:1.0 | 3:1.5 | | | |
| Upstream 2 | prior (0-4) | 0:0.001 | 1:0.015 | 2:0.978 | 3:0.005 | 4:0.0005 |
| Upstream 2 | sd (2) | 0.0073 | | | | |
| Intergenic | shift | 0.966 | | | | |
| Intergenic | mean (2, 3) | 2:1.0 | 3:1.476 | | | |
| Intergenic | prior (0-4) | 0:0.012 | 1:0.13 | 2:0.834 | 3:0.023 | 4:0.0005 |
| Intergenic | sd (2) | 0.0077 | | | | |
| Downstream | shift | 1.071 | | | | |
| Downstream | mean (2, 3) | 2:1.0 | 3:1.5 | | | |
| Downstream | prior (0-4) | 0:0.001 | 1:0.01 | 2:0.987 | 3:0.001 | 4:0.0005 |
| Downstream | sd (2) | 0.06 | | | | |

The above table does not cover parameters for all possible copy numbers (CNs). Parameters were populated for copy numbers that are not covered in the above table using the following strategy. The mean value for CN0 was set as 0 and the mean value for CN1 was set as 0.5. The mean value for CN greater or equal to 3 was populated based on the steps between CN3 and CN2. For example, for the intergeneric region, the CN0 had a mean of 0, CN1 had a mean of 0.5, CN4 had a mean of 1.952, CN6 had mean of 2.428, and so on. The priors for the copy numbers that are not covered in the above table were also populated. The prior for the copy numbers that are not covered in the above table were uniformly distributed. A gmm parameter digital file was created which stored the standard deviation for CN2. The sd values for the other CN states were derived from the standard deviation for CN2. The sd for CN=0 was arbitrarily set at 0.032. The sd for any CN=x was set as the value for CN2 multiplied by the square root of x/2. Values were populated as described for CN=0-10 (11 states), based on the low likelihood that samples have copy number above 10.

### Example 2

In the following example, the methods and systems of determining a HBA1/2 copy number variant genotype as described herein were tested on 3,201 samples from the 1000 Genome Project. Sequence reads were determined from the nucleic acid samples by Illumina® short read technology. Sequence reads which align to about 3,000 pre-determined 2 kb diploid regions within the genome were counted.

Sequence reads which align to four target regions adjacent to the locations of the HBA1 and HBA2 genes in the human genome were also counted. The median alignment MAPQ score for each of the four target regions was 60. The four target regions included a first upstream region upstream of the HBA2 gene and the HBA1 gene, flanking the segmental duplication region X upstream of the HBA2 gene, with the coordinates chr16:167503-169503 in reference genome hg38. The four target regions also included a second upstream region upstream of the HBA2 gene and the HBA1 gene, flanking the segmental duplication region Z upstream of the HBA2 gene, with the coordinates chr16:170263-171875 in reference genome hg38. The four target regions also included an intergenic region in between the HBA2 and HBA1 genes, flanking the segmental duplication region Z upstream of the HBA1 gene, with the coordinates chr16:174519-175845 in reference genome hg38. Finally, the four target regions included a downstream region downstream of the HBA2 and HBA1 genes, with the coordinates chr16:178002-180501 in reference genome hg38.

The sequence read count for each of the target regions was normalized by region length and GC-corrected using the count of the sequence reads aligned to the about 3,000 2 kb diploid regions to obtain a float copy number for each of the four target regions. After determining the normalized and GC corrected depth, the final copy numbers (CNs) for the four target regions were determined using a Gaussian mixture model (GMM) with the parameters (shift, prior, mean, and sd) defined in Example 1. The normalized and GC corrected depth was first scaled by a shift value that corrects for alignment bias between target regions and the 3000 normalization regions. The posterior probability of CN=i given scaled depth was then computed for i=0-6 based on the pre-trained mean, sd, and prior values from the Gaussian mixture model listed in Example 1. The CN with highest posterior probability was then selected as candidate for the final copy number estimate. The copy number estimated was only determined if the posterior probability was greater than 0.95 and the p-value of scaled depth in the Gaussian distribution of the candidate CN was greater than 0.001.

Then, based on the estimated integer copy numbers for each of the four regions, a HBA1/2 copy number variant genotype was determined for each sample according to Table 1. A copy number genotype was not determined if any one of the four target regions did not have a copy number value above the quality check cutoffs (NA in the table below).

The methods and systems described herein were able to determine a HBA1/2 copy number variant genotype for 3,154/3,201 samples (98.5% of the samples). The proportion of the genotypes determined among the samples is represented in the table below. In the table below, interpretation is research use only (RUO).

TABLE 4

| Genotype | RUO Interpretation | Count | Proportion |
|---|---|---|---|
| aaa^{3.7}/aa | alpha-globin triplication | 66 | 2.1% |
| aaa^{4.2}/aa | alpha-globin triplication | 8 | 0.2% |
| aa/aa | Normal | 2611 | 81.6% |
| -a^{3.7}/aa | Silent Carrier | 39 | 12.2% |
| -a^{4.2}/aa | Silent Carrier | 14 | 0.4% |
| --/aaa^{3.7} | Carrier* | 1 | 0.03% |
| --/aaa^{4.2} | Carrier* | 0 | 0% |
| -a^{3.7}/-a^{3.7} | Carrier | 41 | 1.3% |
| -a^{4.2}/-a^{4.2} | Carrier | 0 | 0% |
| -a^{3.7}/-a^{4.2} | Carrier | 1 | 0.03% |
| --/aa | Carrier | 21 | 0.7% |
| --/-a^{3.7} | HbH | 0 | 0% |
| --/-a^{4.2} | HbH | 0 | 0% |
| --/-- | Hb Bart's | 0 | 0% |
| NA | NA | 47 | 1.5% |

### Example 3

In the following example, concordance analysis was performed between samples sequenced with both Illumina® short reads and PacBio® orthogonal long reads.

246 cell line samples from the 1000 Genome Project were sequenced with both Illumina® and PacBio® sequencing systems to produce whole genome sequencing (WGS) data. The Illumina® sequence reads were used in a HBA targeted caller method of determining a HBA1/2 copy number variant genotype as described in Example 2.

The below table describes concordance analysis between the HBA targeted caller and orthogonal long read technology. In the table below, “negative” refers to an aa/aa genotype, while “positive” refers to any deletion or duplication call. For concordance, both genotype and specific deletion and/or duplication had to match.

TABLE 5

| | | HBA Targeted Caller | | | |
|---|---|---|---|---|---|
| | | | | No | |
| | | Positive | Negative | Call | |
| Long | Positive | 46 | 0 | 0 | PPA: 100% |
| Read | Negative | 0 | 196 | 4 | NPA: 98% |
| Calls | | PPV: 100% | NPV: 100% | | |

In the table above, “PPV” refers to positive predictive value, “NPV” refers to negative predictive value, “PPA” refers to positive percent agreement, and “NPA” refers to negative percent agreement. As shown in Table 4, the positive predictive value for the HBA targeted caller is 100%.

The following table is a concordance matrix between the HBA targeted caller and orthogonal results separated by copy number genotypes.

TABLE 6

| | | HBA Targeted Caller | | | | | |
|---|---|---|---|---|---|---|---|
| | | | One- | Two-copy | Two-copy | | |
| | | | copy | deletion in | deletion in | | |
| | | Typical | deletion | cis | trans | Duplication | No Call |
| Long | Typical | 196 | 0 | 0 | 0 | 0 | 4 |
| Read | One-copy | 0 | 33 | 0 | 0 | 0 | 0 |
| Calls | deletion | | | | | | |
| | Two-copy | 0 | 0 | 4 | 0 | 0 | 0 |
| | deletion in | | | | | | |
| | cis | | | | | | |
| | Two-copy | 0 | 0 | 0 | 4 | 0 | 0 |
| | deletion in | | | | | | |
| | trans | | | | | | |
| | Duplication | 0 | 0 | 0 | 0 | 5 | 0 |

### Example 4

In the following example, concordance analysis was performed between samples sequenced with both Illumina® short reads and PacBio® orthogonal long reads.

246 cell line samples from the 1000 Genome Project were sequenced with both Illumina® and PacBio® sequencing systems to produce whole genome sequencing (WGS) data. The Illumina® sequence reads were used in another small variant and copy number variant calling method not targeted to HBA.

The below table describes concordance analysis between the non-targeted caller and orthogonal long read technology. In the table below, “negative” refers to an aa/aa genotype, while “positive” refers to any deletion or duplication call. For concordance, both genotype and specific deletion and/or duplication had to match.

TABLE 7

| | | Non-Targeted Caller | | | |
|---|---|---|---|---|---|
| | | | | No | |
| | | Positive | Negative | Call | |
| Long Read | Positive | 4 | 42 | 0 | PPA: 9% |
| Calls | Negative | 0 | 200 | 0 | NPA: 100% |
| | | PPV: 100% | NPV: 83% | | |

In the table above, “PPV” refers to positive predictive value, “NPV” refers to negative predictive value, “PPA” refers to positive percent agreement, and “NPA” refers to negative percent agreement. As shown in Table 6, the positive predictive value for the non-targeted caller is 9%.

The following table is a concordance matrix between the non-targeted caller and orthogonal results separated by copy number genotypes.

TABLE 8

| | | Non-Targeted Caller | | | | | |
|---|---|---|---|---|---|---|---|
| | | | One- | Two-copy | Two-copy | | |
| | | | copy | deletion in | deletion in | | |
| | | Typical | deletion | cis | trans | Duplication | No Call |
| Long | Typical | 200 | 0 | 0 | 0 | 0 | 0 |
| Read | One-copy | 0 | 0 | 0 | 0 | 0 | 33 |
| Calls | deletion | | | | | | |
| | Two-copy | 0 | 0 | 4 | 0 | 0 | 0 |
| | deletion in | | | | | | |
| | cis | | | | | | |
| | Two-copy | 0 | 0 | 0 | 0 | 0 | 4 |
| | deletion in | | | | | | |
| | trans | | | | | | |
| | Duplication | 0 | 0 | 0 | 0 | 0 | 5 |

### Example 5

In the following example, the systems and methods of the methods and systems of determining a HBA1/2 copy number variant genotype as described in Example 2 were tested on 575 trios from the 1000 Genomes Project with no missing calls. In 575/575 trios, the child genotype call was consistent with parent genotype calls. All trio genotypes had copy number calls consistent with Mendelian inheritance.

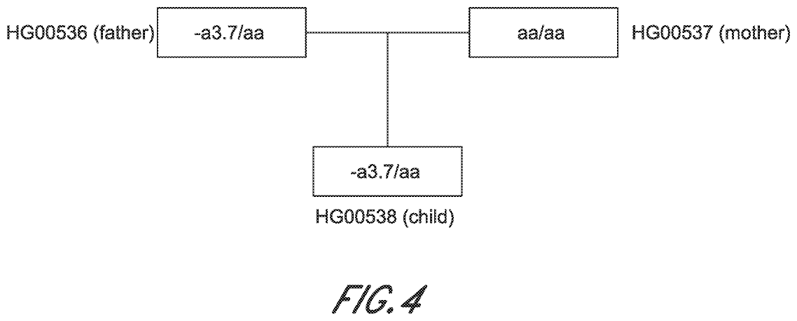

For example, in the trio shown in FIG. 4, the father sample HG00536 was determined to have a -a3.7/aa genotype, while the mother sample HG00537 was determined to have an aa/aa genotype. The child sample HG00538 was determined to have an -a3.7/aa genotype, apparently having inherited an -a3.7 copy from the father and an aa copy from the mother. Thus, the child genotype was consistent with Mendelian inheritance pat terns in the trio shown in FIG. 4 and in the other trios tested.

### Other Considerations

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, such as a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (such as X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (such as a measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

The scope of the present disclosure is not intended to be limited by the specific disclosures of examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A computer-implemented method of determining a HBA1/2 copy number variant genotype in a nucleic acid sample, the method comprising:
determining sequence reads from the nucleic acid sample;
counting sequence reads which align to diploid regions in a human genome within the nucleic acid sample;
counting sequence reads which align to a target region of one or more target regions adjacent to the locations of a HBA1 gene and a HBA2 gene in the human genome; and
determining a HBA1/2 copy number variant genotype based on the count of the sequence reads which align to a target region of the one or more target regions as compared to the count of the sequence reads which align to the diploid regions in the human genome.

2. The method of claim 1, wherein determining a HBA1/2 copy number variant genotype comprises estimating an integer copy number for each of the one or more target regions.

3. The method of claim 2, wherein determining a HBA1/2 copy number variant genotype comprises normalizing the count of the sequence reads which align to each target region by the count of the sequence reads which align to the diploid regions in the human genome to determine a float copy number for each of the one or more target regions.

4. The method of claim 3, wherein estimating an integer copy number for each of the one or more target regions further comprises applying a Gaussian mixture model to the float copy number of the sequence reads which align to each target region.

5. The method of claim 4, wherein the Gaussian mixture model comprises a pre-defined shift, prior, mean, or standard deviation as set forth in Table 3.

6. The method of claim 1, wherein the one or more target regions adjacent to the locations of the HBA1 and HBA2 genes in the human genome comprise a first upstream region upstream of the HBA2 gene and the HBA1 gene.

7. The method of claim 6, wherein the one or more target regions adjacent to the locations of the HBA1 and HBA2 genes in the human genome further comprise a second upstream region upstream of the HBA2 gene and the HBA1 gene.

8. The method of claim 1, wherein the one or more target regions adjacent to the locations of the HBA1 and HBA2 genes in the human genome comprise an intergenic region in between the HBA2 and HBA1 genes, or a downstream region downstream of the HBA2 and HBA1 genes.

9. The method of claim 1, wherein the one or more target regions comprise a first and second upstream region upstream of the HBA2 gene and the HBA1 gene, an intergenic region in between the HBA2 and HBA1 genes, and a downstream region downstream of the HBA2 and HBA1 genes.

10. The method of claim 1, wherein sequence reads align to each of the one or more target regions with an alignment MAPQ score of at least 30.

11. The method of claim 6, wherein the first upstream region flanks a segmental duplication region X upstream of the HBA2 gene.

12. The method claim 7, wherein the second upstream region corresponds to a region within an α4.2 deletion event.

13. The method of claim 7, wherein the second upstream region flanks a segmental duplication region Z upstream of the HBA2 gene.

14. The method of claim 8, wherein the intergenic region corresponds to a region within an α3.7 deletion event.

15. The method of claim 8, wherein the intergenic region flanks a segmental duplication region Z upstream of the HBA1 gene.

16. The method of claim 9, wherein the first upstream region, the second upstream region, the intergenic region, and the downstream region correspond to regions within a deletion event in cis of both HBA1 and HBA2.

17. The method of claim 6, wherein the first upstream region has the coordinates chr16:167503-169503 in reference genome hg38, the second upstream region has the coordinates chr16:170263-171875 in reference genome hg38, the intergenic region has the coordinates chr16:174519-175845 in reference genome hg38, or the downstream region has the coordinates chr16:178002-180501 in reference genome hg38.

18. The method of claim 1, wherein determining a HBA1/2 copy number variant genotype comprises determining an aaa^{3.7}/aa genotype, an aaa^{4.2}/aa genotype, an aa/aa genotype, an -a^{3.7}/aa genotype, an -a^{4.2}/aa genotype, an --/aaa^{3.7} genotype, an --/aaa^{4.2} genotype, an -a^{3.7}/-a^{3.7} genotype, an -a^{4.2}/-a^{4.2} genotype, an -a^{3.7}/-a^{4.2} genotype, an --/aa genotype, an --/a^{3.7} genotype, an --/a^{4.2} genotype, or a --/-- genotype.

19. A computer-implemented method of detecting one or more single-nucleotide variants or indels in a HBA1/2 region in a nucleic acid sample, the method comprising:
determining sequence reads from the nucleic acid sample;
obtaining sequence reads which align to a site of a single-nucleotide variant or indel in a HBA1 gene or a HBA2 gene of a human genome in the nucleic acid sample;
counting sequence reads which contain a base corresponding to an alternative allele at the site of the single-nucleotide variant or indel, wherein counting sequence reads comprises counting sequence reads which align to the HBA1 gene and sequence reads which align to the HBA2 gene; and
creating a digital file including a variant call corresponding to the single-nucleotide variant or indel, wherein the variant call is not specific to the HBA1 gene or the HBA2 gene.

20. The method of claim 19, wherein the single-nucleotide variant or indel comprises HBA2_c.60del, HBA2_c.69C>T, HBA2_c.95+2_95+6delTGAGG, HBA2_c.95+1G>A, HBA1_c.179G>A, HBA2_c.377T>C, HBA2_c.427T>C, HBA2_c.427T>G, HBA2_c.429A>T, HBA2_c.*92A>G, HBA2_c.428A>C, HBA2_c.314G>A, HBA2_c.379G>A, HBA2_c.179G>A, HBA2_c.75T>G, HBA1_c.96-1G>A, HBA1_c.358C>T, or HBA2_c.*94A>G.

21. An electronic system for determining a HBA1/2 copy number variant genotype in a nucleic acid sample comprising a processor configured to perform a method comprising:
determining sequence reads from the nucleic acid sample;
counting sequence reads which align to diploid regions in a human genome within the nucleic acid sample;
counting sequence reads which align to a target region of one or more target regions adjacent to the locations of a HBA1 gene and a HBA2 gene in the human genome; and
determining a HBA1/2 copy number variant genotype based on the count of the sequence reads which align to a target region of the one or more target regions as compared to the count of the sequence reads which align to the diploid regions in the human genome.

22. The electronic system of claim 21, wherein determining a HBA1/2 copy number variant genotype comprises estimating an integer copy number for each of the one or more target regions.

23. The electronic system of claim 22, wherein determining a HBA1/2 copy number variant genotype comprises normalizing the count of the sequence reads which align to each target region by the count of the sequence reads which align to the diploid regions in the human genome to determine a float copy number for each of the one or more target regions.

24. The electronic system of claim 23, wherein estimating an integer copy number for each of the one or more target regions further comprises applying a Gaussian mixture model to the float copy number of the sequence reads which align to each target region.
